(19) United States
(12) Patent Application Publication
(10) Pub. No.: US 2017/0283525 A1
(43) Pub. Date: Oct. 5, 2017

(54) METHODS OF PRODUCING A POLYOLEFIN WAX PRODUCT

(71) Applicant: Honeywell International Inc.; Mail Stop 4D3, Morris Plains, NJ (US)

(72) Inventors: Xiangmin Li, Mendham, NJ (US); Robert C. Mulvaney, III, Flanders, NJ (US); John Clay, Randolph, NJ (US)

(73) Assignee: Honeywell International Inc.; Mail Stop 4D3, Morris Plains, NJ (US)

(21) Appl. No.: 15/444,245

(22) Filed: Feb. 27, 2017

Related U.S. Application Data
(60) Provisional application No. 62/316,033, filed on Mar. 31, 2016.

(51) Int. Cl. C08F 8/50; C07C 4/04

(52) U.S. Cl. CPC C08F 8/50; C07C 4/04

(57) ABSTRACT

Methods of producing a polyolefin wax product are provided herein. In an embodiments, a method of producing a polyolefin wax product includes providing a cracked polyolefin vapor phase and a polyolefin liquid phase that are derived from a recycled polyolefin feedstock having a higher number average molecular weight than the polyolefin wax product. A portion of the cracked polyolefin vapor phase is condensed at a temperature and pressure above a boiling point of olefins having less than or equal to 12 carbon atoms to produce the polyolefin wax product.

### CROSS-REFERENCE TO RELATED APPLICATIONS

This application claims the benefit of U.S. Provisional Application No. 62/316,033 filed Mar. 31, 2016.

### TECHNICAL FIELD

The technical field generally relates to methods of forming a polyolefin wax product, and more particularly relates to methods of processing a recycled polyolefin feedstock to form a polyolefin wax product with minimal discoloration and malodor.

### BACKGROUND

Waxes are broadly divided into several well established groups including paraffin waxes (normally obtained from petroleum oil lubricating distillates), microcrystalline wax (usually obtained from residual lubricating oil fractions), and polyolefin waxes (typically manufactured from direct polymerization of olefin monomers such as ethylene or from thermal degradation of polyolefin resins such as polyethylene). Each of these wax types has been found to have specific physical properties making them especially attractive for particular utilities. For example, polyolefin waxes such as polyethylene waxes are often used in the formulation of colorants for plastics, in polyvinyl chloride lubricants, in adhesives, and in inks and coatings to decrease friction. Polyethylene waxes may further be used as release agents or as slip agents.

Polyolefin waxes have a variety of structures and properties. For example, polyethylene waxes have number average molecular weights in the range of about 1500 grams per mole (g/mol) to about 20,000 g/mol. High grade polyolefin waxes may be obtained by the controlled polymerization of an olefin (e.g., ethylene, propylene, and the like) to obtain desired properties in terms of molecular weight, melting point, viscosity and hardness. Recently, there has been a rise in the use of lower grade polyolefin waxes that are typically derived from the thermal decomposition of recycled polyolefin feedstock, particularly recycled polyethylenes such as low density polyethylene (LDPE), high density polyethylene (HDPE), linear low density polyethylene (LLDPE), polypropylene, and the like. Such lower grade products have become popular for use in application or locations where product quality is secondary to cost. However, polyolefin waxes derived from the thermal decomposition of recycled polyolefin feedstock are typically inferior in quality, exhibit undesirable discoloration, and often emit undesirable odors.

Prior efforts to minimize discoloration and malodor of the polyolefin waxes derived from the thermal decomposition of recycled polyolefin feedstock have involved pre-washing of the recycled polyolefin feedstock and post-treating the polyolefin wax product to remove odor-causing species. However, such techniques add process stages, thereby increasing cost, and are only marginally effective at reducing discoloration and malodor of the resulting polyolefin wax product. Another effort to minimize discoloration and malodor of the polyolefin waxes derived from the thermal decomposition of recycled polyolefin feedstock has involved cracking the recycled polyethylene feedstock in a reducing atmosphere, i.e., in the presence of a reducing species in the vapor phase. However, while such techniques yield polyolefins of reduced discoloration, further reductions in discoloration are still desirable.

Accordingly, it is desirable to provide methods of forming polyolefin wax from a recycled polyolefin feedstock with minimized discoloration and malodor. It is also desirable to provide such methods that can be carried out without pre-washing the recycled polyolefin feedstock and that can also be carried out in the absence of a deodorizing step while still minimizing discoloration and malodor. Furthermore, other desirable features and characteristics will become apparent from the subsequent detailed description and the appended claims, taken in conjunction with the accompanying drawings and the foregoing technical field and background.

### BRIEF SUMMARY

Methods of producing a polyolefin wax product are provided herein. In an embodiment, a method of producing a polyolefin wax product includes providing a cracked polyolefin vapor phase and a polyolefin liquid phase that are derived from a recycled polyolefin feedstock having a higher number average molecular weight than the polyolefin wax product. A portion of the cracked polyolefin vapor phase is condensed at a temperature and pressure above a boiling point of olefins having less than or equal to 12 carbon atoms to produce the polyolefin wax product.

In another embodiment, a method of producing a polyolefin wax product includes heating a recycled polyolefin feedstock on an evaporative surface within a short path distillation device. The recycled polyolefin feedstock is heated at a cracking temperature that is sufficient to thermally depolymerize polyolefins in the recycled polyolefin feedstock and that is further sufficient to evaporate the resulting cracked polyolefin to produce a cracked polyolefin vapor phase. The cracked polyolefin vapor phase is separate from a polyolefin liquid phase. A portion of the cracked polyolefin vapor phase is condensed on a condensation surface within the short path distillation device to produce the polyolefin wax product.

In another embodiment, a method of producing a polyolefin wax product includes heating a recycled polyolefin feedstock at a cracking temperature of from about 350 to about 450° C. and a pressure of less than or equal to about 20 KPa to produce a cracked polyolefin vapor phase that is separate from a polyolefin liquid phase. A portion of the cracked polyolefin vapor phase is condensed at a temperature of from about 100 to about 180° C. and at a pressure of from about 0.0001 to about 20 KPa to produce the polyolefin wax product.

### BRIEF DESCRIPTION OF THE DRAWINGS

The various embodiments will hereinafter be described in conjunction with the following drawing figures, wherein like numerals denote like elements, and wherein:

FIG. 1 is a schematic diagram of a method and apparatus for producing a polyolefin wax in accordance with an exemplary embodiment as described herein; and

FIG. 2 is a graph showing relationships between boiling point and number of carbon atoms of hydrocarbons at different pressures.

### DETAILED DESCRIPTION

The following detailed description is merely exemplary in nature and is not intended to limit the methods and apparatuses for producing a polyolefin wax as claimed. Furthermore, there is no intention to be bound by any theory presented in the preceding background or the following detailed description.

Methods of producing polyolefin waxes from a recycled polyolefin feedstock with minimized discoloration are provided herein. In particular, a portion of a cracked polyolefin vapor phase that is derived from a recycled polyolefin feedstock is condensed at a temperature and pressure above a boiling point of olefins having less than or equal to 12 carbon atoms to produce the polyolefin wax product. The “cracked polyolefin vapor phase,” as described herein, is a vapor phase containing the cracked product of the recycled polyolefin feedstock and may contain, among other species, olefins having a lower number average molecular weight (Mn) than the polyolefins of the recycled polyolefin feedstock. The “recycled polyolefin feedstock,” as described herein, is recovered from prior consumer or industrial use and represents waste material that was previously produced as an end or intermediate product from a different production process. The “polyolefin wax product,” as referred to herein, includes thermally cracked polyolefin compounds of lower polydispersity than the feedstock. In embodiments, the polyolefin wax product has a number average molecular weight (Mn) of from about 400 grams per mole (g/mol) to about 7000 g/mol, for example from about 1500 g/mol to about 4000 g/mol. Without being bound by theory, it is believed that by obtaining the polyolefin wax product from a portion of the cracked polyolefin vapor phase and by condensing the portion of the cracked polyolefin vapor phase at the temperature and pressure that are above the boiling point of olefins having less than or equal to 12 carbon atoms, most color and odor-causing species are separated from the desired species (color-causing species generally remain in the liquid phase, while odor-causing species remain in the vapor phase after condensation), while desirable species from the cracked polyolefin vapor phase are condensed and yield a desirable polyolefin wax product that exhibits minimized discoloration and malodor. By condensing the portion of the cracked polyolefin vapor phase at the recited conditions, pre-washing of the recycled polyolefin feedstock can be avoided while still minimizing discoloration and malodor. Likewise, a deodorizing step is also unnecessary.

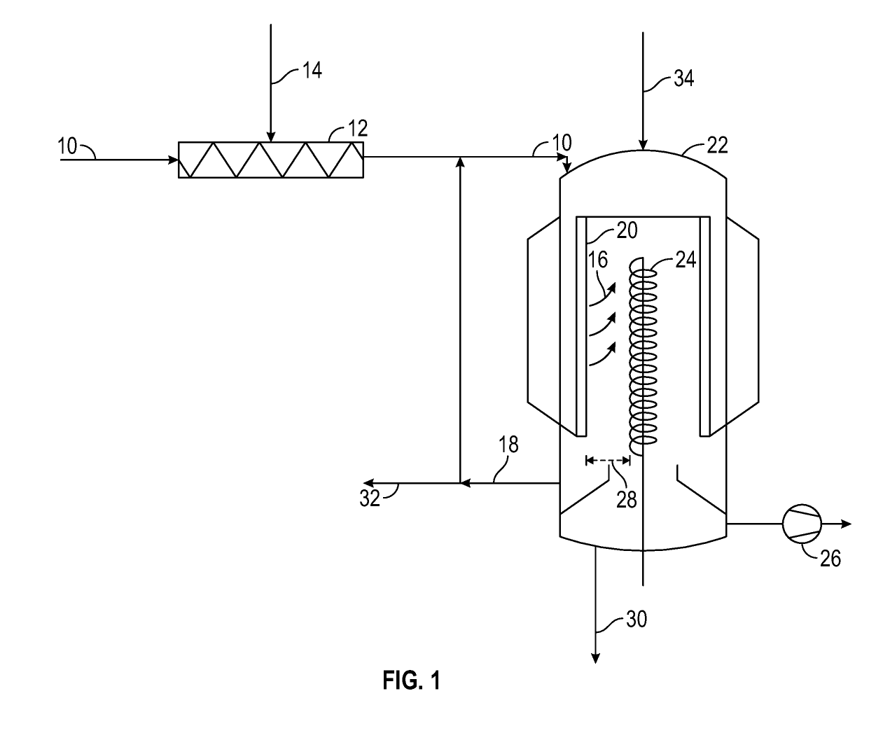

Referring to FIG. 1, an exemplary method of and apparatus for producing a polyolefin wax product is shown. In the embodiment shown, a continuous process is employed. However, in other embodiments, it is to be appreciated that the methods as described herein may be conducted in batch processes. In embodiments, a recycled polyolefin feedstock 10 is provided. The recycled polyolefin feedstock 10 comprises thermoplastic polyolefins such as polyethylenes, for example polyethylenes with the formula (C₂H₄)ₙH₂ where n is typically from about 100 to about 1000, and polypropylenes, such as polypropylenes with the formula (C₃H₆)ₘH₂, where m is typically from about 50 to about 800, or a mixture of thermoplastic polyolefins. Specific examples of suitable polyethylenes include low density polyethylene (LDPE), high density polyethylene (HDPE), linear low density polyethylene (LLDPE), and the like. In an exemplary embodiment, the recycled polyolefin feedstock 10 is linear low density polyethylene (LLDPE) plastic waste. For example, the recycled polyolefin feedstock 10 may be polyethylene films or bags from post-consumer recycle or post-industrial recycle processes. The recycled polyolefin feedstock 10 may include common impurities, such as one or more of dirt, plasticizers, antioxidants, or fillers. The presence of such impurities does not materially diminish the desired properties of the polyolefin wax product 30 that is ultimately obtained through the methods described herein.

In embodiments, the recycled polyolefin feedstock 10 may be pre-processed in preparation for thermal decomposition. For example, although not shown, the recycled polyolefin feedstock 10 can be physically modified, for example, by chopping, shredding, or pelletizing. Notably, while the recycled polyolefin feedstock 10 may be pre-processed to obtain a more suitable physical form for thermal decomposition, the polyolefin wax product 30 may be produced in the absence of pre-washing the recycled polyolefin feedstock 10, which would ordinarily be desirable for removing some of the impurities. However, because the impurities do not materially diminish the desired properties of the polyolefin wax product 30, pre-washing is not necessary in accordance with the methods described herein.

In embodiments and as shown in FIG. 1, the recycled polyolefin feedstock 10 may be pre-cracked in a pre-cracking vessel 12. “Pre-cracking,” as referred to herein, is an initial cracking unit operation whereby the recycled polyolefin feedstock 10 is subject to processing conditions that are sufficient to crack or thermally degrade polyolefins in the recycled polyolefin feedstock 10, albeit with all products of the pre-cracking vessel 12 discharged in a single stream 16, before further cracking is conducting in a downstream unit operation. Pre-cracking can be conducted in various types of vessels and/or conduits provided that adequate residence time and temperatures are achieved to effectuate cracking. In embodiments, the pre-cracking vessel 12 is an extruder 12, and the recycled polyolefin feedstock 10 is fed to the extruder 12 with pre-cracking conducted in the extruder 12. In embodiments, the recycled polyolefin feedstock 10 is heated in the pre-cracking vessel 12 to a temperature of from about 350 to about 450 ° C., with residence time of the recycled polyolefin feedstock 10 in the pre-cracking vessel 12 sufficiently long to enable thermal degradation of olefin species in the recycled polyolefin feedstock 10. Temperatures recited herein refer to internal temperatures of the material being heated and not atmospheric temperatures, while pressures recited herein refer to pressure surrounding the material being treated. The recycled polyolefin feedstock 10 is optionally compounded with fresh polyolefin feedstock 14. In embodiments, it is to be appreciated that the recycled polyolefin feedstock 10 may be provided directly to the extruder 12 in the absence of pre-processing depending upon the physical form of the recycled polyolefin feedstock 10. Further, in embodiments, it is to be appreciated that the recycled polyolefin feedstock 10 may be provided to the extruder 12 for melting the recycled polyolefin feedstock 10 at sub-cracking conditions, i.e., without pre-cracking occurring in the extruder 12.

After optional pre-cracking, the recycled polyolefin feedstock 10 is heated at a cracking temperature that is sufficient to thermally depolymerize polyolefins in the recycled polyolefin feedstock 10 and that is further sufficient to evaporate the resulting cracked polyolefin to produce a cracked polyolefin vapor phase 16 separate from a polyolefin liquid phase 18. As referred to here, in the polyolefin liquid phase 18 is the liquid portion left over after yielding the cracked polyolefin vapor phase 16 from the recycled polyolefin feedstock 10. In embodiments and as shown in FIG. 1, the recycled polyolefin feedstock 10 is heated on an evaporative surface 20 to evaporate the cracked polyolefin. In embodiments, the evaporative surface 20 is heated using a heating element chosen from fused salts, an induction heating element, or a resistive heating element, which enable higher temperatures of the evaporative surface 20 to be achieved than conventional oil heating mechanisms. As set forth in further detail below, the evaporative surface 20 may be contained within a short path distillation (SPD) device 22, although it is to be appreciated that the recycled polyolefin feedstock 10 may be heated at the cracking temperature in alternative devices such as an autoclave or a thin film evaporator.

As with optional pre-cracking, the recycled polyolefin feedstock 10 may be heated at a cracking temperature of from about 350 to about 450° C. to effectuate thermal degradation of polyolefin species contained therein. In embodiments, the recycled polyolefin feedstock 10 is heated at the cracking temperature and at reduced pressures that are less than atmospheric pressure. In particular, in embodiments, the recycled polyolefin feedstock 10 is heated at the cracking temperature and at a reduced pressure of less than or equal to about 20 KPa, such as from about 0.0001 to about 2.0 KPa, or such as from about 0.0001 to about 0.2 KPa, or such as from about 0.0001 to about 0.13 KPa, to produce the cracked polyolefin vapor phase 16 separate from the polyolefin liquid phase 18. More particularly, the cracked polyolefin vapor phase 16 is produced concurrently with cracking of the recycled polyolefin feedstock 10. The reduced pressure impacts content of the cracked polyolefin vapor phase 16. Without being bound by theory, polyolefins of higher molecular weight have a lower estimated vapor pressure and by heating the recycled polyolefin feedstock 10 at the cracking temperature and at reduced pressures, a greater content of polyolefin/paraffin species having number average molecular weights within the desired range for the polyolefin wax product 30 are evaporated into the cracked polyolefin vapor phase 16. For example, polyolefin and paraffin species having about 100 carbon atoms (and a Mn ˜1400 g/mol) have an estimated vapor pressure of about 0.5 KPa (as estimated using COSMOTherm Thermodynamics software), while polyolefin and paraffin species having about 40 carbon atoms have an estimated vapor pressure of about 9 KPa (as determined from the Design Institute for Physical Properties database). Further, it has been found that by heating the recycled polyolefin feedstock 10 at the cracking temperature and at reduced pressures, a higher number average molecular weight is observed in the polyolefin wax product 30 as compared to circumstances in which the recycled polyolefin feedstock 10 is heated at the cracking temperature and at ambient pressures. The higher number average molecular weight corresponds to a shifted distribution of thermally cracked polyolefin species of different individual Mn in the polyolefin wax product 30 and, thus, provides a product with lower content of undesired lower Mn species.

In embodiments, to further inhibit formation of color-causing species, the recycled polyolefin feedstock 10 is heated in the presence of a reducing atmosphere, i.e., a reducing species in the vapor phase. For example, hydrogen 34 may be introduced into the ambient surrounding the evaporative surface 20, along with inert gas such as nitrogen, to produce the reducing atmosphere. While it is to be appreciated that hydrogen 34 content of the reducing atmosphere may depend upon volume of the device 22 within which the recycled polyolefin feedstock 10 is heated, in embodiments, hydrogen 34 is provided in an amount of from about 10⁻⁷ to about 10⁻² m3/kg of the recycled polyolefin feedstock 10. Although not shown, it is to be appreciated that hydrogen may be introduced into the optional pre-cracking vessel 12 as well, along with inert gas such as nitrogen, to produce the reducing atmosphere.

A portion of the cracked polyolefin vapor phase 16 is condensed at a temperature and pressure that is above a boiling point of olefins having less than or equal to 12 carbon atoms. In embodiments, the portion of the cracked polyolefin vapor phase 16 is condensed at a temperature and pressure that is above a boiling point of olefins having less than or equal to 30 carbon atoms. By condensing the portion of the cracked polyolefin vapor phase 16 under such conditions, co-condensation of the carbon-containing species with less than 12 carbon atoms (or less than 30 carbon atoms, depending upon the condensation conditions) is minimized and such species remain in the vapor phase, effectively excluding such species from the polyolefin wax product 30. The carbon-containing species with less than 12 carbon atoms are typically responsible for undesirable malodor of the polyolefin wax product 30.

In embodiments and referring to FIG. 1, the portion of the cracked polyolefin vapor phase 16 is condensed on a condensation surface 24, such as a cooling coil using and appropriate coolant. As set forth in further detail below, the condensation surface 24 may be contained within the short path distillation (SPD) device 22, although it is to be appreciated that the portion of the cracked polyolefin vapor phase 16 may be condensed in alternative devices such as a dedicated condensation unit that is distinct from the device within which evaporation is conducted.

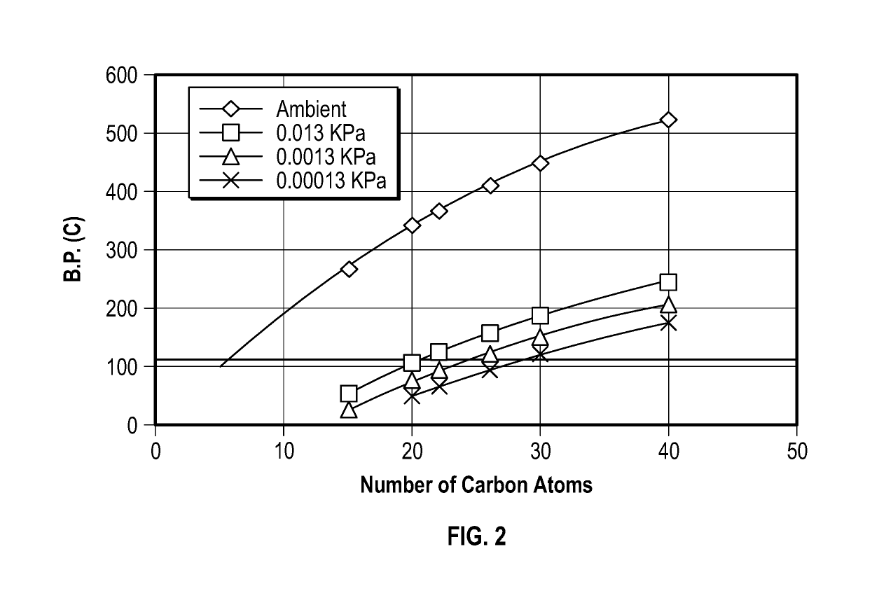

Referring to FIG. 2, relationships between boiling point and number of carbon atoms of hydrocarbons are shown at different pressures. Typical condensation stages employ a coolant such as water for cooling the condensation surface 24, with temperatures of the condensation surface 24 typically from about 100 to about 180° C. As can be seen from FIG. 2, hydrocarbons having as few as from 6 to 9 carbon atoms have boiling points at atmospheric pressure that may be below the aforementioned operating temperatures. For example, at an operating temperature of 120° C. and at atmospheric pressure, the conditions are above the boiling point of hydrocarbons with 6 or less carbon atoms such that those species can be substantially maintained in the vapor phase and stripped out. However, the conditions are below the boiling point of hydrocarbons having greater than 7 carbon atoms such that those species are included in the condensate. Because hydrocarbons having less than 12 carbon atoms are often responsible for malodor in the resulting polyolefin wax product 30, further deodorizing processing would ordinarily be required for condensate obtained at an operating temperature of 120° C. and at atmospheric pressure.

In embodiments, the portion of the cracked polyolefin vapor phase 16 is condensed at a pressure of less than or equal to about 20 KPa, such as from about 0.0001 to about 2.0 KPa, or such as from about 0.0001 to about 0.13 KPa, or such as from about 0.0001 to about 0.02 KPa, to produce the polyolefin wax product 30 as a condensate. As can be seen from FIG. 2, as operating pressures decrease, effective boiling points of hydrocarbon species are decreased. By decreasing the operating pressure at which condensation is conducted, undesirable polyolefins and paraffins having less than 12 carbon atoms can be substantially excluded from the polyolefin wax product 30 and stripped off with the remaining vapor phase while still condensing at a temperature of from about 100 to about 180° C., which is typical when hot oil is used as the coolant. For example, an operating pressure of less than about 20 KPa and an operating temperature range of from about 100 to about 180° C. during condensing will enable polyolefins and paraffins having less than 12 carbon atoms to remain in the vapor phase. Thus, in embodiments, the condensate may be taken as the polyolefin wax product 30 in the absence of a further deodorizing stage.

In embodiments, the recycled polyolefin feedstock 10 is heated at the cracking temperature and at substantially the same pressure at which the portion of the cracked polyolefin vapor phase 16 is condensed to thereby exploit the aforementioned dynamics of both evaporating and condensing at the reduced pressures. In embodiments, the portion of the cracked polyolefin vapor phase 16 is directly condensed after heating the recycled polyolefin feedstock 10 to thermally depolymerize the polyolefins in the recycled polyolefin feedstock 10 and evaporate the resulting cracked polyolefin. In this regard, heating the recycled polyolefin feedstock 10 at the cracking temperature and condensing the portion of the cracked polyolefin vapor phase 16 may be conducted in the same distillation vessel to enable a unitary vacuum system 26 to be used to provide the desired operating pressure. By directly condensing the portion of the cracked polyolefin vapor phase 16 after heating, optionally in the same distillation vessel, drastic pressure drops can be avoided that would render process implementation difficult. As alluded to above, in embodiments, heating the recycled polyolefin feedstock 10 at the cracking temperature and condensing the portion of the cracked polyolefin vapor phase 16 is conducted in the SPD device 22, which provides a minimal distance 28 between the evaporative surface 20 and the condensation surface 24 to thereby minimize pressure drop between evaporative and condensation areas. The particular distance 28 between the evaporative surface 20 and the condensation surface 24 is dependent upon the scale of the SPD device as well as pre-determined operational vapor flow rate, which determinations are conventionally made when engineering SPD devices. The distance 28, as referred to herein, is the closest point between any portion of the evaporative surface 20 and any portion of the condensation surface 24.

After condensing the portion of the cracked polyolefin vapor phase 16, the condensate may be taken as the polyolefin wax product 30 and the polyolefin liquid phase 18 remains separate from the polyolefin wax product 30. In embodiments, residence time of the recycled polyolefin feedstock 10 on the evaporative surface 20 may be relatively short, ranging from tens of seconds to minutes (such as from about 30 seconds to about 5 minutes). As such, cracking of the polyolefins in the recycled polyolefin feedstock 10 per pass may be relatively low. While pre-cracking may assist with conversion per pass efficiency, in embodiments, the polyolefin liquid phase 18 is recycled to combine with the recycled polyolefin feedstock 10. In embodiments, the polyolefin liquid phase 18 and the recycled polyolefin feedstock 10 are combined after pre-cracking the recycled polyolefin feedstock 10, although it is to be appreciated that in other embodiments, the polyolefin liquid phase 18 and the recycled polyolefin feedstock 10 may be combined at other stages. In embodiments, a drag stream 32 can be removed from the polyolefin liquid phase 18 to minimize buildup of impurities and color-causing species within the process.

The following Examples are illustrative of the methods of producing polyolefin wax as described herein and are not intended to be limiting.

### EXAMPLES

Various examples of polyolefin wax products were prepared employing an autoclave (for comparative purposes) and a vacuum evaporator device (to illustrate the embodiments as described herein).

### Comparative Example 1

A comparative polyolefin wax product was prepared in the autoclave using a recycled polyethylene feedstock. The recycled polyethylene feedstock was thermally cracked at a temperature of about 400° C. for a period of about 45 minutes. A liquid phase in the autoclave was collected after cracking and taken as a polyethylene wax product. The polyethylene wax product was gray in color as determined through visual inspection.

### Examples 1 and 2

Exemplary polyolefin wax products were prepared using a recycled polyethylene feedstock with both pre-cracking in an autoclave and without pre-cracking, with subsequent heating and condensation using the vacuum evaporator device. Pre-cracking was conducted in an autoclave at an operating temperature of about 400° C. and a residence time of about 30 minutes. Exemplary polyolefin wax products were prepared with the evaporative surface of the vacuum evaporator device operated at a temperature of about 410° C. and with a residence time of the recycled polyethylene feedstock in the vacuum evaporator device of about 2 minutes. Referring to TABLE I, process dynamics and results are shown for preparation of the polyolefin wax products, with Example 1 prepared without pre-cracking and with Example 2 prepared with pre-cracking.

TABLE I

| | Example 1 | Example 2 |
|---|---|---|
| Recycled Polyethylene Feedstock, Mn | ~23,000 | ~6,000 |
| Evaporative Surface Temp, ° C. | 410 | 410 |
| Residence Time, Min | 2 | 2 |
| Per Pass Conversion in Evaporator | <2% | ~8% |
| Product Color | White | White |

### Examples 3-5

Additional exemplary polyolefin wax products were prepared in a similar manner to Example 1, but with the recycled polyethylene feedstock evaporated from the evaporative surface of the vacuum evaporator at different pressures. The recycled polyethylene feedstock has a Mn of ˜23,000 g/mol and a Referring to TABLE II, Mn and polydispersity index (Mw/Mn) are shown for polyolefin wax products formed when the recycled polyethylene feedstock is evaporated from the evaporative surface of the vacuum evaporator at different pressures.

TABLE II

| | Mn | PDI |
|---|---|---|
| Ex. 3, Evap @ Ambient Pressure | 429 | 1.17 |
| Ex. 4, Evap @ 0.13 Kpa | 1,800 | 1.22 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration in any way. Rather, the foregoing detailed description will provide those skilled in the art with a convenient road map for implementing an exemplary embodiment. It being understood that various changes may be made in the function and arrangement of elements described in an exemplary embodiment without departing from the scope as set forth in the appended claims.

What is claimed is:

1. A method of producing a polyolefin wax product, wherein the method comprises:
providing a cracked polyolefin vapor phase and a polyolefin liquid phase derived from a recycled polyolefin feedstock having a higher number average molecular weight than the polyolefin wax product; and
condensing a portion of the cracked polyolefin vapor phase at a temperature and pressure above a boiling point of olefins having less than or equal to 12 carbon atoms to produce the polyolefin wax product.

2. The method of claim 1, wherein providing the cracked polyolefin vapor phase and the polyolefin liquid phase comprises heating the recycled polyolefin feedstock at a cracking temperature sufficient to thermally depolymerize polyolefins in the recycled polyolefin feedstock and evaporate the resulting cracked polyolefin to produce the cracked polyolefin vapor phase separate from the polyolefin liquid phase.

3. The method of claim 2, wherein the recycled polyolefin feedstock is heated at the cracking temperature and at substantially the same pressure at which the portion of the cracked polyolefin vapor phase is condensed.

4. The method of claim 3, wherein heating the recycled polyolefin feedstock at the cracking temperature and condensing the portion of the cracked polyolefin vapor phase is conducted in the same distillation vessel.

5. The method of claim 4, wherein heating the recycled polyolefin feedstock at the cracking temperature and condensing the portion of the cracked polyolefin vapor phase is conducted in a short path distillation device.

6. The method of claim 4, further comprising pre-cracking the recycled polyolefin feedstock in a pre-cracking vessel prior to heating the recycled polyolefin feedstock at the cracking temperature in the distillation vessel.

7. The method of claim 6, wherein pre-cracking the recycled polyolefin feedstock comprises feeding the recycled polyolefin feedstock to an extruder, and wherein the recycled polyolefin feedstock is pre-cracked in the extruder.

8. The method of claim 2, wherein the portion of the cracked polyolefin vapor phase is directly condensed after heating the recycled polyolefin feedstock to thermally depolymerize the polyolefins in the recycled polyolefin feedstock and evaporate the resulting cracked polyolefin.

9. The method of claim 2, further comprising recycling the polyolefin liquid phase to combine with the recycled polyolefin feedstock.

10. The method of claim 2, wherein the recycled polyolefin feedstock is heated in the presence of a reducing species in the vapor phase.

11. The method of claim 1, wherein the portion of the cracked polyolefin vapor phase is condensed at a pressure of from about 0.0001 to about 20 Kpa.

12. The method of claim 11, wherein the portion of the cracked polyolefin vapor phase is condensed at a temperature of from about 100 to about 180° C.

13. The method of claim 1, further comprising packing the condensed portion of the cracked polyolefin vapor phase in the absence of a further deodorizing stage.

14. The method of claim 1, wherein the cracked polyolefin vapor phase and the polyolefin liquid phase are derived from the recycled polyolefin feedstock comprising one or more of dirt, plasticizers, antioxidants, or fillers.

15. The method of claim 14, wherein the cracked polyolefin vapor phase and the polyolefin liquid phase are derived from the recycled polyolefin feedstock in the absence of pre-washing the recycled polyolefin feedstock.

16. A method of producing a polyolefin wax product, wherein the method comprises:
heating a recycled polyolefin feedstock on an evaporative surface within a short path distillation device at a cracking temperature sufficient to thermally depolymerize polyolefins in the recycled polyolefin feedstock and evaporate the resulting cracked polyolefin to produce a cracked polyolefin vapor phase separate from a polyolefin liquid phase; and
condensing a portion of the cracked polyolefin vapor phase on a condensation surface within the short path distillation device to produce the polyolefin wax product.

17. The method of claim 16, wherein the recycled polyolefin feedstock is heated within the short path distillation device employing a heating element chosen from fused salts, an induction heating element, or a resistive heating element.

18. A method of producing a polyolefin wax product, wherein the method comprises:
heating a recycled polyolefin feedstock at a cracking temperature of from about 350 to about 450° C. and a pressure of less than or equal to about 20 KPa to produce a cracked polyolefin vapor phase separate from a polyolefin liquid phase; and
condensing a portion of the cracked polyolefin vapor phase at a temperature of from about 100 to about 180° C. and at a pressure of from about 0.0001 to about 20 Kpa to produce the polyolefin wax product.
